(19) United States
(12) Patent Application Publication
(10) Pub. No.: US 2023/0080718 A1
(43) Pub. Date: Mar. 16, 2023

(54) SILICON PRECURSOR MATERIALS, SILICON-CONTAINING FILMS, AND RELATED METHODS

(71) Applicant: ENTEGRIS, INC., Billerica, MA (US)

(72) Inventors: YoonHae Kim, Suwon-si (KR); Sungsil Cho, Anyang-si (KR); HwanSoo Kim, Hwaseong-si (KR); KieJin Park, Suwon-si (KR)

(21) Appl. No.: 17/859,953

(22) Filed: Jul. 7, 2022

Related U.S. Application Data
(60) Provisional application No. 63/238,542, filed on Aug. 30, 2021.

(51) Int. Cl. C01B 33/027; C01B 33/08; C07F 7/02; C07F 7/18; C23C 16/40; C23C 16/34

(52) U.S. Cl. CPC C01B 33/027; C01B 33/08; C07F 7/025; C07F 7/18; C23C 16/401; C23C 16/345

(57) ABSTRACT

Some embodiments relate to a method for depositing a silicon precursor on a substrate. The method comprises obtaining a silicon precursor material comprising at least one siloxane linkage, and obtaining at least one co-reactant precursor material. The silicon precursor material is volatized to obtain a silicon precursor vapor. The at least one co-reactant precursor material is volatized to obtain at least one co-reactant precursor vapor. The silicon precursor vapor and the at least one co-reactant precursor vapor are contacted with the substrate, under chemical vapor deposition conditions, sufficient to form a silicon-containing film on a surface of the substrate. Some embodiments relate to silicon precursor materials for chemical vapor deposition.

### FIELD

This disclosure generally relates to silicon precursor materials, silicon-containing films, and related methods.

### BACKGROUND

Tetraethylorthosilicate (TEOS) is used as a precursor in low-temperature vapor deposition processes to form thin films of silicon dioxide (e.g., SiO₂). Low-temperature vapor deposition processes are performed at temperatures below 200° C. TEOS is not a suitable precursor for the formation of silicon-containing thin films via high-temperature vapor deposition processes.

### SUMMARY

Some embodiments relate to a precursor for chemical vapor deposition. In some embodiments, the precursor for chemical vapor deposition may comprise, consist of, or consist essentially of a silicon precursor material. In some embodiments, the silicon precursor material may comprise, consist of, or consist essentially of a compound of formula:(A¹A²A³)Si—O— Si(B¹B²B³), wherein each of A¹, A², A³, B¹, B², and B³ is independently a hydrogen, a halide, an alkyl, a cycloalkyl, an alkoxy, an amino, an alkylamino, an aminoalkyl, an ethynyl, an phenyl, an allyl, a vinyl, or an acetoxy. In some embodiments, the silicon precursor material may have a reactivity with at least one co-reactant precursor material, under chemical vapor deposition conditions, sufficient to result in a silicon-containing film as a reaction product.

Some embodiments relate to a method for depositing a silicon precursor on a substrate. In some embodiments, the method may comprise, consist of, or consist essentially of one or more of the following steps: obtaining a silicon precursor material comprising at least one siloxane linkage; obtaining at least one co-reactant precursor material; volatizing the silicon precursor material to obtain a silicon precursor vapor; volatizing the at least one co-reactant precursor material to obtain at least one co-reactant precursor vapor; and contacting the silicon precursor vapor and the at least one co-reactant precursor vapor with the substrate, under chemical vapor deposition conditions, sufficient to form a silicon-containing film on a surface of the substrate.

### BRIEF DESCRIPTION OF THE DRAWINGS

Reference is made to the drawings that form a part of this disclosure, and which illustrate embodiments in which the materials and methods described herein can be practiced.

FIG. 1 is a flowchart of a method for depositing a silicon precursor on a substrate, according to some embodiments of the present disclosure.

FIG. 2 is a schematic diagram of a silicon-containing article, according to some embodiments of the present disclosure.

FIG. 3 is a graphical view of deposition rates of bis(diethylamino)-1,1,3,3-tetramethyldisiloxane (BDEA-TMDSO) compared to tetraethylorthosilicate (TEOS) at 560° C. and 650° C., according to some embodiments of the present disclosure.

### DETAILED DESCRIPTION

As used herein, the term “siloxane linkage” refers to linkages of the formula: —(Si—O—Si)_{n—}, wherein n is 1 to 6. In some embodiments, one or more siloxane linkages may associate to form a siloxane compound. In some embodiments, the siloxane compound may comprise, consist of, or consist essentially of, or may be selected from the group consisting of, at least one of a disiloxane, an oligosiloxane, a cyclosiloxane, a polysiloxane, or any combination thereof. In some embodiments, where two or more siloxane linkages are present, the siloxane linkages may share one or more silicon atoms.

As used herein, the term “alkyl” refers to a hydrocarbon chain radical having from 1 to 30 carbon atoms. The alkyl may be attached via a single bond. An alkyl having n carbon atoms may be designated as a “Cₙ alkyl.” For example, a “C₃ alkyl” may include n-propyl and isopropyl. An alkyl having a range of carbon atoms, such as 1 to 30 carbon atoms, may be designated as a C₁-C₃₀ alkyl. In some embodiments, the alkyl is saturated (e.g., single bonds). In some embodiments, the alkyl is unsaturated (e.g., double bonds and/or triple bonds). In some embodiments, the alkyl is linear. In some embodiments, the alkyl is branched. In some embodiments, the alkyl is substituted. In some embodiments, the alkyl is unsubstituted. In some embodiments, the alkyl may comprise, consist of, or consist essentially of, or may be selected from the group consisting of, at least one of a C₁-C₁₂ alkyl, a C₁-C₁₁ alkyl, a C₁-C₁₀ alkyl, a C₁-C₉ alkyl, a C₁-C₈ alkyl, a C₁-C₇ alkyl, a C₁-C₆ alkyl, a C₁-C₄ alkyl, a C₁-C₃ alkyl, or any combination thereof. In some embodiments, the alkyl may comprise, consist of, or consist essentially of, or may be selected from the group consisting of, at least one of methyl, ethyl, n-propyl, 1-methylethyl (isopropyl), n-butyl, iso-butyl, sec-butyl, n-pentyl, 1,1-dimethylethyl (t-butyl), n-pentyl, iso-pentyl, n-hexyl, isohexyl, 3-methylhexyl, 2-methylhexyl, octyl, decyl, dodecyl, octadecyl, or any combination thereof.

As used herein, the term “cycloalkyl” refers to a non-aromatic carbocyclic ring radical attached via a single bond and having from 3 to 8 carbon atoms in the ring. The term includes a monocyclic non-aromatic carbocyclic ring and a polycyclic non-aromatic carbocyclic ring. For example, two or more cycloalkyls may be fused, bridged, or fused and bridged to obtain the polycyclic non-aromatic carbocyclic ring. In some embodiments, the cycloalkyl may comprise, consist of, or consist essentially of, or may be selected from the group consisting of, at least one of cyclopropyl, cyclobutyl, cyclopentyl, cyclohexyl, cycloheptyl, cyclooctyl, or any combination thereof.

As used herein, the term “alkoxy” refers to a radical of formula —OR, wherein R is an alkyl, as defined herein. In some embodiments, the alkoxy may comprise, consist of, or consist essentially of, or may selected from the group consisting of, at least one of methoxy, ethoxy, methoxy, ethoxy, n-propoxy, 1-methylethoxy (isopropoxy), n-butoxy, iso-butoxy, sec-butoxy, tert-butoxy, or any combination thereof.

As used herein, the term “amine,” “alkylamino,” and the like refer to a radical of formula —N(R^{a}R^{b}R^{c}), wherein each of R^{a}, R^{b}, and R^{c} is independently a hydrogen or an alkyl, as defined herein. In some embodiments, the term “amine” includes an amino, as defined herein. In some embodiments, the amine may comprise, consist of, or consist essentially of a primary amine, a secondary amine, a tertiary amine, or a quaternary amine. In some embodiments, the amine may comprise, consist of, or consist essentially of an alkyl amine, a dialkylamine, or a trialkyl amine. In some embodiments, the amine may comprise, consist of, or consist essentially of, or may be selected from the group consisting of, at least one of methyl amine, dimethylamine, ethylamine, diethylamine, isopropylamine, di-isopropylamine, butylamine, sec-butylamine, tert-butylamine, di-sec-butylamine, isobutylamine, di-isobutylamine, di-tert-pentylamine, ethylmethylamine, isopropyl-n-propylamine, or any combination thereof. Examples of the alkylamines may include, without limitation, one or more of the following: primary alkylamines, such as, for example and without limitation, methylamine, ethylamine, n-propylamine, isopropylamine, n-butylamine, sec-butylamine, isobutylamine, t-butylamine, pentylamine, 2-aminopentane, 3-aminopentane, 1-amino-2-methylbutane, 2-amino-2-methylbutane, 3-amino-2-methylbutane, 4-amino-2-methylbutane, hexylamine, 5-amino-2-methylpentane, heptylamine, octylamine, nonylamine, decylamine, undecylamine, dodecylamine, tridecylamine, tetradecylamine, pentadecylamine, hexadecylamine, heptadecylamine, and octadecylamine; secondary alkylamines, such as, for example and without limitation, dimethylamine, diethylamine, dipropylamine, diisopropylamine, dibutylamine, diisobutylamine, di-sec-butylamine, di-t-butylamine, dipentylamine, dihexylamine, diheptylamine, dioctylamine, dinonylamine, didecylamine, methylethylamine, methylpropylamine, methylisopropylamine, methylbutylamine, methylisobutylamine, methyl-sec-butylamine, methyl-t-butylamine, methylamylamine, methylisoamylamine, ethylpropylamine, ethylisopropylamine, ethylbutylamine, ethylisobutylamine, ethyl-sec-butylamine, ethylamine, ethylisoamylamine, propylbutylamine, and propylisobutylamine; and tertiary alkylamines, such as, for example and without limitation, trimethylamine, triethylamine, tripropylamine, tributylamine, tripentylamine, dimethylethylamine, methyldiethylamine, and methyldipropylamine. Examples of polyamines may include, without limitation, one or more of the following: ethylenediamine, propylenediamine, trimethylenediamine, tetramethylenediamine, 1,3-diaminobutane, 2,3-diaminobutane, pentamethylenediamine, 2,4-diaminopentane, hexamethylenediamine, heptamethylenediamine, octamethylenediamine, nonamethylenediamine, N-methylethylenediamine, N,N-dimethylethylenediamine, trimethylethylenediamine, N-ethylethylenediamine, N,N-diethylethylenediamine, triethylethylenediamine, 1,2,3-triaminopropane, hydrazine, tris(2-aminoethyl) amine, tetra(aminomethyl)methane, diethylenetriamine, triethylenetetramine, tetraethylpentamine, heptaethyleneoctamine, nonaethylenedecamine, and diazabicyloundecene.

As used herein, the term “halide” refers to a —Cl, —Br, —I, or —F.

As used herein, the term “ethynyl” refers to —C≡CH.

As used herein, the term “phenyl” refers to —C₆H₅.

As used herein, the term “amino” refers to —NH₂.

As used herein, the term “allyl” refers to —CH₂CH═CH₂.

As used herein, the term “vinyl” refers to —CH═CH₂.

As used herein, the term “acetoxy” refers to —OC(═O)CH₃.

Some embodiments relate to silicon precursor materials. The silicon precursor materials may exhibit one or more of improved thermal stability at high temperatures (e.g., such as, temperatures of 500° C. or greater) and improved thin film deposition rates (e.g., such as, deposition rates of two (2) times greater than deposition rates of conventional precursor materials). The silicon precursor materials may exhibit improved performance in high-temperature chemical vapor deposition processes. Further advantages of the silicon precursor materials of the present disclosure may include, without limitation, one or more of improved step coverage at low pressures, improved step coverage at high temperatures, and reduced amount of impurities, among others. The silicon precursor materials may be used in high-temperature chemical vapor deposition (CVD) processes to improve the quality of silicon-containing films resulting therefrom.

In some embodiments, the silicon precursor material may comprise, consist of, or consist essentially of one or more siloxane linkages. For example, in some embodiments, the silicon precursor material may comprise, consist of, or consist essentially of one siloxane linkage, two siloxane linkages, three siloxane linkages, or four or more siloxane linkages.

In some embodiments, the silicon precursor material may comprise, consist of, or consist essentially of a compound of formula:

(A¹A²A³)Si—O—Si(B¹B²B³),

wherein each of A¹, A², A³, B¹, B², and B³ is independently a hydrogen, a halide, an alkyl, a cycloalkyl, an alkoxy, an amino, an alkylamino, an aminoalkyl, an ethynyl, an phenyl, an allyl, a vinyl, or an acetoxy.

In some embodiments, the silicon precursor material may comprise, consist of, or consist essentially of, or may be selected from the group consisting of, at least one of the following: bis(diethylamino)-1,1,3,3-tetramethyldisiloxane (BDEA-TMDSO), 1,3-bis(isopropylamino)tetramethyldisiloxane (BIPA-TMDSO), hexamethyldisiloxane (HMDS 0), 1,3-diphenyl-1,3-dimethyldisiloxane, 1,1,3,3-tetramethyldisiloxane, 1,1,1-triethyl-3,3-dimethyldisiloxane, 1,1,3,3-tetra-n-octyldimethyldisiloxane, bis(nonafluorohexyl)tetramethyldisiloxane, 1,3-bis(trifluoropropyl)tetramethyldisiloxane, 1,3-di-n-butyltetramethyldisiloxane, 1,3-di-n-octyltetramethyldisiloxane, 1,3-diethyltetramethyldisiloxane, 1,3-diphenyltetramethyl-disiloxane, hexa-n-butyldisiloxane, hexaethyldisiloxane, hexavinyldisiloxane, 1,1,1,3,3-pentamethyl-3-acetoxydisiloxane, 1-allyl-1,1,3,3-tetramethyldisiloxane, 1,3-bis (3-aminopropyl)tetramethyl disiloxane, 1,3-bis (heptadecafluoro-1,1,2,2-tetrahydrodecyl)-tetramethyldisiloxane, 1,3-divinyltetraphenyldisiloxane, 1,3-divinyltetramethyl-disiloxane, 1,3-diallyltetrakis (trimethyl siloxy)disiloxane, 1,3-diallyltetramethyl-disiloxane, 1,3-diphenyltetrakis (dimethylsiloxy)disiloxane, (3-chloropropyl)pentamethyl-disiloxane, 1,3-divinyltetrakis (trimethyl siloxy)disiloxane, 1,1,3,3-tetraisopropyl-disiloxane, 1,1,3,3-tetravinyldimethyldisiloxane, 1,1,3,3-tetracyclopentyldichloro-disiloxane, vinylpentamethyldisiloxane, 1,3-bis (3-chloroisobutyl)tetramethyldisiloxane, hexaphenyldisiloxane, 1,1,1-triethyl-3,3,3-trimethyldisiloxane, 1,3-bis(chloromethyl)tetramethyldisiloxane, 1,1,3,3-tetramethyl-1,3-diethoxy disiloxane, 1,1,3,3-tetraphenyldimethyldisiloxane, methacryloxypentamethyl-disiloxane, pentamethyldisiloxane, 1,3-bis (3-chloropropyl)tetramethyldisiloxane, 1,3-bis (4-hydroxybutyl)tetramethyldisiloxane, 1,3-bis (triethoxysilylethyl)tetramethyl-disiloxane, 3-aminopropylpentamethyldisiloxane, 1,3-dichloro-1,3-diphenyl-1,3-dimethyldisiloxane, 1,3-diethynyltetramethyldisiloxane, n-butyl-1,1,3,3-tetramethyldisiloxane, 1,3-dichlorotetraphenyldisiloxane, 1,3-dichlorotetramethyl-disiloxane, 1,3-di-t-butyldisiloxane, 1,3-dimethyltetramethoxydisiloxane, 1,3-divinyltetraethoxydisiloxane, 1,1,3,3-tetraethoxy-1,3-dimethyldisiloxane, vinyl-1,1,3,3-tetramethyldisiloxane, platinum-[1,3-bis(cyclohexyl)imidazol-2-ylidene divinyltetramethyldisiloxane, hexachlorodisiloxane (HCDSO), 1,1,3,3-tetraisopropyl-1-chlorodisiloxane, 1, 1,1-trimethyl-3,3,3-triphenyldisiloxane, 1,3-bis(trimethylsiloxy)-1,3-dimethyldisiloxane, 3,3-diphenyl-tetramethyltrisiloxane, 3-phenylheptamethyltrisiloxane, hexamethylcyclotrisiloxane, n-propylheptamethyltrisiloxane, 1,5-diethoxyhexamethyltrisiloxane, 3-ethylheptamethyl-trisiloxane, 3-(tetrahydrofurfuryloxypropyl)heptamethyltrisiloxane, 3-(3,3,3-trifluoropropyl)heptamethyltrisiloxane, 1,1,3,5,5-pentaphenyl-1,3,5-trimethyltrisiloxane, octamethyltrisiloxane, 1,1,5,5-tetraphenyl-1,3,3,5-tetramethyltrisiloxane, hexaphenylcyclotrisiloxane, 1,1,1,5,5,5-hexamethyltrisiloxane, octachlorotrisiloxane, 3-phenyl-1,1,3,5,5-pentamethyltrisiloxane, (3,3,3-trifluoropropyl)methylcyclotrisiloxane, 1,3,5-trivinyl-1,1,3,5,5-pentamethyltrisiloxane, 1,3,5-trivinyl-1,3,5-trimethylcyclotrisiloxane, 3-(m-pentadecylphenoxypropyl)heptamethyltrisiloxane, limonenyltrisiloxane, 3-dodecylheptamethyltrisiloxane, 3-octylheptamethyltrisiloxane, 1,3,5-triphenyltrimethylcyclotrisiloxane, 1,1,1,3,3,5,5-heptamethyltrisiloxane, 1,1,3,3,5,5-hexamethyltrisiloxane, 1,1,1,5,5,5-hexaethyl-3-methyltrisiloxane, 1,5-dichlorohexamethyltri siloxane, 3-(3-hydroxypropyl)heptamethyltrisiloxane, hexamethylcyclomethylphosphonoxytrisiloxane, 3-octadecylheptamethyltrisiloxane, tetrakis(dimethylsiloxy)silane, 1,1,3,3,5,5,7,7-octamethyltetrasiloxane, a diphenyl siloxane-dimethylsiloxane copolymer, 1,3-diphenyl-1,3-dimethyldisiloxane, octamethylcyclotetrasiloxane, 1,3-bis(trimethylsiloxy)-1,3-dimethyldisiloxane, a dimethylsiloxane-[65-70% (60% propylene oxide/40% ethylene oxide)]block copolymer, bis(hydroxypropyl)tetramethyldisiloxane, tetra-n-propyltetramethylcyclotetrasiloxane, octaethylcyclotetrasiloxane, decamethyltetrasiloxane, dodecamethylcyclohexasiloxane, dodecamethylpentasiloxane, tetradecamethylhexasiloxane, hexaphenylcyclotrisiloxane, polydimethylsiloxane, polyoctadecylmethylsiloxane, hexacosyl terminated polydimethylsiloxane, decamethylcyclopentasiloxane, poly(3,3,3-trifluoropropylmethylsiloxane), trimethylsiloxy terminated polydimethylsiloxane, 1,1,3,3,5,5,7,7,9,9-decamethylpentasiloxane, triethylsiloxy terminated polydiethylsiloxane, or any combination thereof.

The silicon precursor materials may have a reactivity with at least one co-reactant precursor material, under chemical vapor deposition conditions, sufficient to result in a silicon-containing film as a reaction product.

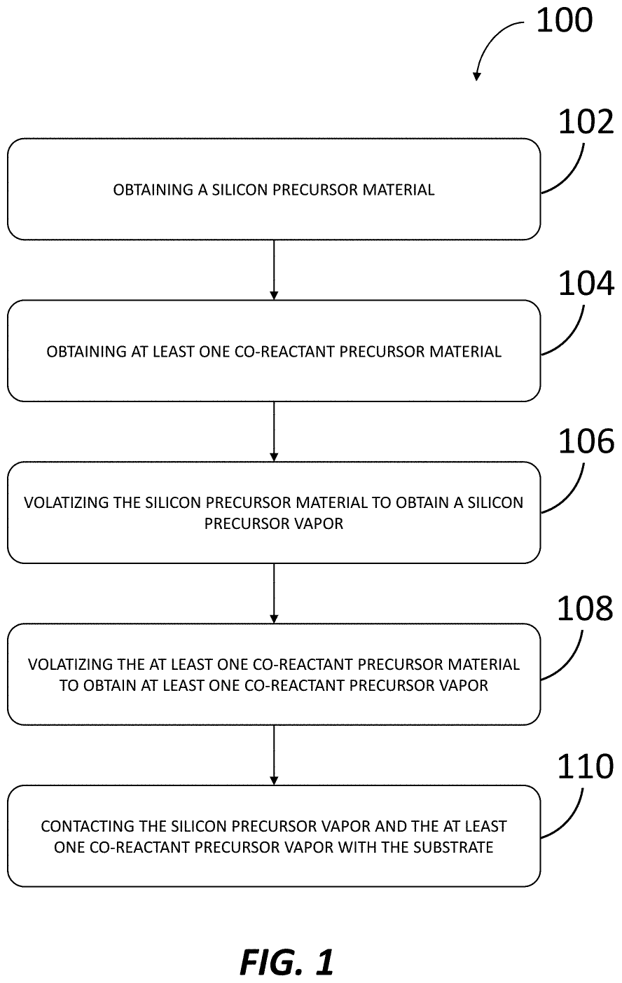

FIG. 1 is a flowchart of a method for depositing a silicon precursor on a substrate, according to some embodiments of the present disclosure. As shown in FIG. 1, the method 100 may comprise, consist of, or consist essentially of one or more of the following steps: a step 102 of obtaining a silicon precursor material; a step 104 of obtaining at least one co-reactant precursor material; a step 106 of volatizing the silicon precursor material to obtain a silicon precursor vapor; a step 108 of volatizing the at least one co-reactant precursor material to obtain at least one co-reactant precursor vapor; and a step 110 of contacting the silicon precursor vapor and the at least one co-reactant precursor vapor with the substrate, under chemical vapor deposition conditions, sufficient to form a silicon-containing film on a surface of the substrate.

The step 102 may comprise, consist of, or consist essentially of obtaining a silicon precursor material. The silicon precursor material may comprise, consist of, or consist essentially of any one or more of the silicon precursor materials disclosed herein. The obtaining may comprise obtaining a container or other vessel comprising the silicon precursor material. In some embodiments, the silicon precursor material may be obtained in a container or other vessel in which the silicon precursor material may be vaporized.

The step 104 may comprise, consist of, or consist essentially of obtaining at least one co-reactant precursor material. The at least one co-reactant precursor material may be selected to obtain a desired silicon-containing film. In some embodiments, the desired silicon-containing film may comprise, consist of, or consist essentially of at least one of silicon nitride, silicon oxide, or any combination thereof. In some embodiments, the at least one co-reactant precursor material may comprise, consist of, or consist essentially of at least one of N₂, H₂, NH₃, N₂H₄, CH₃HNNH₂, CH₃HNNHCH₃, NCH₃H₂, NCH₃CH₂H₂, N(CH₃)₂H, N(CH₃CH₂)₂H, N(CH₃)₃, N(CH₃CH₂)₃, Si(CH₃)₂NH, pyrazoline, pyridine, ethylene diamine, a radical thereof, or any combination thereof. In some embodiments, the at least one co-reactant precursor material may comprise, consist of, or consist essentially of at least one of H₂, O₂, O₃, H₂O, H₂O₂, NO, N₂O, NO₂, CO, CO₂, a carboxylic acid, an alcohol, a diol, a radical thereof, or any combination thereof. The obtaining may comprise obtaining a container or other vessel comprising the at least one co-reactant precursor material. In some embodiments, the at least one co-reactant precursor material may be obtained in a container or other vessel in which the at least one co-reactant precursor material may be vaporized.

The step 106 may comprise, consist of, or consist essentially of volatizing the silicon precursor material to obtain a silicon precursor vapor. The volatizing may comprise, consist of, or consist essentially of heating the silicon precursor material sufficient to obtain the silicon precursor vapor. In some embodiments, the volatizing may comprise, consist of, or consist essentially of heating a container comprising the silicon precursor material. In some embodiments, the volatizing may comprise, consist of, or consist essentially of heating the silicon precursor material in a deposition chamber in which the chemical vapor deposition process is performed. In some embodiments, the volatizing may comprise, consist of, or consist essentially of heating a conduit for delivering the silicon precursor material, silicon precursor vapor, or any combination thereof to, for example, a deposition chamber. In some embodiments, the volatizing may comprise, consist of, or consist essentially of operating a vapor delivery system comprising the silicon precursor material. In some embodiments, the volatizing may comprise, consist of, or consist essentially of heating to a temperature sufficient to vaporize the silicon precursor material to obtain the silicon precursor vapor. In some embodiments, the volatizing may comprise, consist of, or consist essentially of heating to a temperature below a decomposition temperature of at least one of the silicon precursor material, the silicon precursor vapor, or any combination thereof. In some embodiments, the silicon precursor material may be present in a gas phase, in which case the step 106 is optional and not required. For example, the silicon precursor material may comprise, consist of, or consist essentially of the silicon precursor vapor.

The step 108 may comprise, consist of, or consist essentially of volatizing the at least one co-reactant precursor material to obtain the at least one co-reactant precursor vapor. In some embodiments, the volatizing may comprise, consist of, or consist essentially of heating the at least one co-reactant precursor material sufficient to obtain the at least one co-reactant precursor vapor. In some embodiments, the volatizing may comprise, consist of, or consist essentially of heating a container comprising the at least one co-reactant precursor material. In some embodiments, the volatizing may comprise, consist of, or consist essentially of heating the at least one co-reactant precursor material in a deposition chamber in which the chemical vapor deposition process is performed. In some embodiments, the volatizing may comprise, consist of, or consist essentially of heating a conduit for delivering the at least one co-reactant precursor material, the at least one co-reactant precursor vapor, or any combination thereof to, for example, a deposition chamber. In some embodiments, the volatizing may comprise, consist of, or consist essentially of operating a vapor delivery system comprising the at least one co-reactant precursor material. In some embodiments, the volatizing may comprise, consist of, or consist essentially of heating to a temperature sufficient to vaporize the at least one co-reactant precursor material to obtain the at least one co-reactant precursor vapor. In some embodiments, the volatizing may comprise, consist of, or consist essentially of heating to a temperature below a decomposition temperature of at least one of the at least one co-reactant precursor material, the at least one co-reactant precursor vapor, or any combination thereof. In some embodiments, the at least one co-reactant precursor material may be present in a gas phase, in which case the step 108 is optional and not required. For example, the at least one co-reactant precursor material may comprise, consist of, or consist essentially of the at least one co-reactant precursor vapor.

The step 110 may comprise, consist of, or consist essentially of contacting the silicon precursor vapor and the at least one co-reactant precursor vapor with the substrate, under chemical vapor deposition conditions, sufficient to form a silicon-containing film on a surface of the substrate. The contacting may be performed in any system, apparatus, device, assembly, chamber thereof, or component thereof suitable for chemical vapor deposition processes, including, for example and without limitation, a deposition chamber, among others. The silicon precursor vapor and the at least one co-reactant precursor may be contacted with the substrate at the same time. For example, each of the silicon precursor vapor, the at least one co-reactant precursor vapor, and the substrate may be present in the deposition chamber at the same time. Accordingly, the contacting may comprise contemporaneous contacting or simultaneous contacting of the silicon precursor vapor and the at least one co-reactant precursor vapor with the substrate.

This contemporaneous or simultaneous contacting of at least the silicon precursor vapor, the at least one co-reactant precursor vapor, and the substrate may differentiate the chemical vapor deposition (CVD) process disclosed herein from an atomic layer deposition (ALD) process. In atomic layer deposition, the substrate is alternately and sequentially contacted (e.g., in one or more cycles) with each vapor phase precursor. For example, a first vapor phase precursor may be contacted with a substrate in a chamber, without any other vapor phase precursors present, for a first period of time. Upon removing excess of the first vapor phase precursor from the chamber, a second vapor phase precursor may be contacted with the substrate in the chamber, without any other vapor phase precursors present, for a second period of time. Additional vapor phase precursors may be employed and this process may be repeated in one or more cycles or pulses. In the chemical vapor deposition process disclosed herein, both of the vapor phase precursors may be contacted with the substrate at the same time and therefore, unlike atomic layer deposition processes, are not alternately and sequentially contacted with the substrate. Accordingly, the contacting may not comprise alternately contacting each of the silicon precursor vapor and the at least one co-reactant precursor vapor with the substrate. For example, in some embodiments, the contacting does not comprise sequentially contacting each of the silicon precursor vapor and the at least one co-reactant precursor vapor with the substrate. In some embodiments, the contacting does not comprise alternately and sequentially contacting each of the silicon precursor vapor and the at least one co-reactant precursor vapor with the substrate.

The chemical vapor deposition conditions may comprise, consist of, or consist essentially of a deposition temperature. The deposition temperature may be a temperature less than the thermal decomposition temperature of at least one of the silicon precursor vapor, the at least one co-reactant precursor vapor, or any combination thereof. The deposition temperature may be sufficiently high to reduce or avoid condensation of at least one of the silicon precursor vapor, the at least one co-reactant precursor vapor, or any combination thereof. In some embodiments, the substrate may be heated to the deposition temperature. In some embodiments, the chamber or other vessel in which the substrate is contacted with the silicon precursor vapor and the at least one co-reactant precursor vapor is heated to the deposition temperature. In some embodiments, at least one of the silicon precursor vapor, the at least one co-reactant precursor vapor, or any combination thereof may be heated to the deposition temperature.

The deposition temperature may be a temperature of 200° C. to 2500° C. In some embodiments, the deposition temperature may be a temperature of 500° C. to 700° C. For example, in some embodiments, the deposition temperature may be a temperature of 500° C. to 680° C., 500° C. to 660° C., 500° C. to 640° C., 500° C. to 620° C., 500° C. to 600° C., 500° C. to 580° C., 500° C. to 560° C., 500° C. to 540° C., 500° C. to 520° C., 520° C. to 700° C., 540° C. to 700° C., 560° C. to 700° C., 580° C. to 700° C., 600° C. to 700° C., 620° C. to 700° C., 640° C. to 700° C., 660° C. to 700° C., or 680° C. to 700° C. In other embodiments, the deposition temperature may be a temperature of greater than 200° C. to 2500° C., such as, for example and without limitation, a temperature of 400° C. to 2000, 500° C. to 2000° C., 550° C. to 2400° C., 600° C. to 2400° C., 625° C. to 2400° C., 650° C. to 2400° C., 675° C. to 2400° C., 700° C. to 2400° C., 725° C. to 2400° C., 750° C. to 2400° C., 775° C. to 2400° C., 800° C. to 2400° C., 825° C. to 2400° C., 850° C. to 2400° C., 875° C. to 2400° C., 900° C. to 2400° C., 925° C. to 2400° C., 950° C. to 2400° C., 975° C. to 2400° C., 1000° C. to 2400° C., 1025° C. to 2400° C., 1050° C. to 2400° C., 1075° C. to 2400° C., 1100° C. to 2400° C., 1200° C. to 2400° C., 1300° C. to 2400° C., 1400° C. to 2400° C., 1500° C. to 2400° C., 1600° C. to 2400° C., 1700° C. to 2400° C., 1800° C. to 2400° C., 1900° C. to 2400° C., 2000° C. to 2400° C., 2100° C. to 2400° C., 2200° C. to 2400° C., 2300° C. to 2400° C., 500° C. to 2000° C., 500° C. to 1900° C., 500° C. to 1800° C., 500° C. to 1700° C., 500° C. to 1600° C., 500° C. to 1500° C., 500° C. to 1400° C., 500° C. to 1300° C., 500° C. to 1200° C., 500° C. to 1100° C., 500° C. to 1000° C., 500° C. to 1000° C., 500° C. to 900° C., or 500° C. to 800° C.

The chemical vapor deposition conditions may comprise, consist of, or consist essentially of a deposition pressure. In some embodiments, the deposition pressure may comprise, consist of, or consist essentially of a vapor pressure of at least one of the silicon precursor vapor, the at least one co-reactant precursor vapor, or any combination thereof. In some embodiments, the deposition pressure may comprise, consist of, or consist essentially of a chamber pressure.

The deposition pressure may be a pressure of 0.001 Torr to 100 Torr. For example, in some embodiments, the deposition pressure may be a pressure of 1 Torr to 30 Torr, 1 Torr to 25 Torr, 1 Torr to 20 Torr, 1 Torr to 15 Torr, 1 Torr to 10 Torr, 5 Torr to 50 Torr, 5 Torr to 40 Torr, 5 Torr to 30 Torr, 5 Torr to 20 Torr, or 5 Torr to 15 Torr. In other embodiments, the deposition pressure may be a pressure of 1 Torr to 100 Torr, 5 Torr to 100 Torr, 10 Torr to 100 Torr, 15 Torr to 100 Torr, 20 Torr to 100 Torr, 25 Torr to 100 Torr, 30 Torr to 100 Torr, 35 Torr to 100 Torr, 40 Torr to 100 Torr, 45 Torr to 100 Torr, 50 Torr to 100 Torr, 55 Torr to 100 Torr, 60 Torr to 100 Torr, 65 Torr to 100 Torr, 70 Torr to 100 Torr, 75 Torr to 100 Torr, 80 Torr to 100 Torr, 85 Torr to 100 Torr, 90 Torr to 100 Torr, 95 Torr to 100 Torr, 1 Torr to 95 Torr, 1 Torr to 90 Torr, 1 Torr to 85 Torr, 1 Torr to 80 Torr, 1 Torr to 75 Torr, or 1 Torr to 70 Torr. In other further embodiments, the deposition pressure may be a pressure of 1 mTorr to 100 mTorr, 1 mTorr to 90 mTorr, 1 mTorr to 80 mTorr, 1 mTorr to 70 mTorr, 1 mTorr to 60 mTorr, 1 mTorr to 50 mTorr, 1 mTorr to 40 mTorr, 1 mTorr to 30 mTorr, 1 mTorr to 20 mTorr, 1 mTorr to 10 mTorr, 100 mTorr to 300 mTorr, 150 mTorr to 300 mTorr, 200 mTorr to 300 mTorr, or 150 mTorr to 250 mTorr, or 150 mTorr to 225 mTorr.

The contacting may be sufficient to result in a deposition rate of 1 to 50 nm per minute. For example, in some embodiments, the contacting may be sufficient to result in a deposition rate of 10 to 35 nm per minute, 11 to 35 nm per minute, 12 to 35 nm per minute, 13 to 35 nm per minute, 10 to 32 nm per minute, 11 to 32 nm per minute, 12 to 32 nm per minute, 13 to 32 nm per minute, 15 to 32 nm per minute, 16 to 32 nm per minute, 17 to 32 nm per minute, 18 to 32 nm per minute, 19 to 32 nm per minute, 20 to 32 nm per minute, 21 to 32 nm per minute, 22 to 32 nm per minute, 23 to 32 nm per minute, 24 to 32 nm per minute, 25 to 32 nm per minute, 26 to 32 nm per minute, 27 to 32 nm per minute, 28 to 32 nm per minute, 29 to 32 nm per minute, or 30 to 32 nm per minute. In some embodiments, the deposition rate may be in reference to one or more of a deposition temperature and a deposition pressure, among other chemical vapor deposition conditions. In some embodiments, the contacting may be sufficient to result in a deposition rate of 1 to 1500 nm per minute, 1 to 1400 nm per minute, 1 to 1300 nm per minute, 1 to 1200 nm per minute, 1 to 1100 nm per minute, 1 to 1000 nm per minute, 1 to 900 nm per minute, 1 to 800 nm per minute, 1 to 700 nm per minute, 1 to 600 nm per minute 1 to 500 nm per minute, 1 to 400 nm per minute, 1 to 300 nm per minute, 1 to 200 nm per minute, or 1 to 100 nm per minute.

The contacting may be sufficient to result in a step coverage of 10% to 80%. For example, in some embodiments, the contacting may be sufficient to result in a step coverage of 30% to 45%, 31% to 45%, 32% to 45%, 33% to 45%, 34% to 45%, 35% to 45%, 36% to 45%, 37% to 45%, 38% to 45%, 39% to 45%, or 40% to 45%. In some embodiments, the step coverage may be in reference to one or more of a deposition temperature and a deposition pressure, among other chemical vapor deposition conditions.

The contacting may be sufficient to result in a deposition rate of 1.2 to 5 times greater than a silicon precursor material control, wherein the silicon precursor material control comprises tetraethoxysilane (TEOS). For example, in some embodiments, the contacting may be sufficient to result in a deposition rate of 1.4 to 3 times greater than a silicon precursor material control, 1.5 to 3 times greater than a silicon precursor material control, 1.6 to 3 times greater than a silicon precursor material control, 1.7 to 3 times greater than a silicon precursor material control, 1.8 to 3 times greater than a silicon precursor material control, 1.9 to 3 times greater than a silicon precursor material control, 2 to 3 times greater than a silicon precursor material control, 2.1 to 3 times greater than a silicon precursor material control, 2.2 to 3 times greater than a silicon precursor material control, 2.3 to 3 times greater than a silicon precursor material control, 2.4 to 3 times greater than a silicon precursor material control, 2.5 to 3 times greater than a silicon precursor material control, or 2.6 to 3 times greater than a silicon precursor material control. In some embodiments, the deposition rate may be in reference to one or more of a deposition temperature and a deposition pressure, among other chemical vapor deposition conditions.

The contacting may be sufficient to result in a wet etch rate of 1 to 20 nm per minute. For example, in some embodiments, the contacting may be sufficient to result in a wet etch rate of 1 to 19 nm per minute, 1 to 18 nm per minute, 1 to 17 nm per minute, 1 to 16 nm per minute, 1 to 15 nm per minute, 1 to 14 nm per minute, 1 to 13 nm per minute, 1 to 12 nm per minute, 1 to 11 nm per minute, 1 to 10 nm per minute, 1 to 9 nm per minute, 1 to 8 nm per minute, 1 to 7 nm per minute, 1 to 6 nm per minute, 1 to 5 nm per minute, 1 to 4 nm per minute, 2 to 20 nm per minute, 3 to 20 nm per minute, 4 to 20 nm per minute, 5 to 20 nm per minute, 6 to 20 nm per minute, 7 to 20 nm per minute, 8 to 20 nm per minute, 9 to 20 nm per minute, 10 to 20 nm per minute, 11 to 20 nm per minute, 12 to 20 nm per minute, 13 to 20 nm per minute, 14 to 20 nm per minute, 15 to 20 nm per minute, 16 to 20 nm per minute, 17 to 20 nm per minute, or 18 to 20 nm per minute. In some embodiments, the wet etch rate is based on reference to thermal oxidation at 100:1 HF.

The contacting may be sufficient to result in a thermal shrinkage of less than 10%. For example, in some embodiments, the contacting may be sufficient to result in a thermal shrinkage of 0.1% to 10%, 0.1% to 5%, 0.1% to 5%, 0.1% to 4.8%, 0.1% to 4.6%, 0.1% to 4.4%, 0.1% to 4.2%, 0.1% to 4%, 0.1% to 3.8%, 0.1% to 3.6%, 0.1% to 3.4%, 0.1% to 3.2%, 0.1% to 3%, 0.1% to 2.8%, 0.1% to 2.6%, 0.1% to 2.4%, 0.1% to 2.2%, 0.1% to 2%, 0.1% to 1.8%, 0.1% to 1.6%, 0.1% to 1.4%, 0.1% to 1.2%, 0.1% to 1%, 0.1% to 0.8%, 0.1% to 0.6%, 0.1% to 0.5%, or 0.1% to 0.4%.

The substrate may comprise, consist of, or consist essentially of at least one of Si, Co, Cu, Al, W, WN, WC, TiN, Mo, MoC, SiO₂, W, SiN, WCN, Al₂O₃, AN, ZrO₂, La₂O₃, TaN, RuO₂, IrO₂, Nb₂O₃, Y₂O₃, hafnium oxide, or any combination thereof. In some embodiments, the substrate may comprise other silicon-based substrates, such as, for example, one or more of polysilicon substrates, metallic substrates, and dielectric substrates.

Some embodiments relate to silicon-containing films, such as silicon-containing films prepared according to the method of FIG. 1.

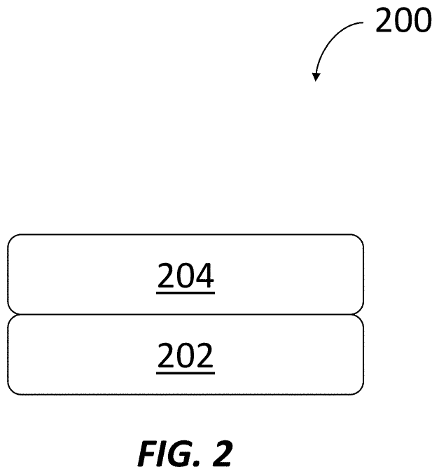

FIG. 2 is a schematic diagram of a silicon-containing article, according to some embodiments of the present disclosure. As shown in FIG. 2, a silicon-containing article 200 may comprise, consist of, or consist essentially of a substrate 202 and a silicon-containing film 204. In the illustrated embodiment, the silicon-containing article 200 comprises a silicon-containing film 204 on at least a portion of the substrate 202.

### Example 1

Silicon Precursor Material

A non-limiting example of a silicon precursor material includes bis(diethylamino)-1,1,3,3-tetramethyldisiloxane (BDEA-TMDSO). The chemical structure of BDEA-TMDSO is presented below:

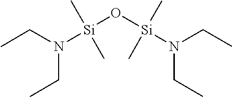 [SMILES: CCN(CC)[Si](C)(C)O[Si](C)(C)N(CC)CC]

### Example 2

Silicon Precursor Material

A non-limiting example of a silicon precursor material includes 1,3-bis(isopropylamino)tetramethyldisiloxane (BIPA-TMDSO). The chemical structure of BIPA-TMDSO is presented below:

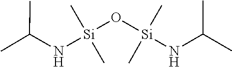

### Example 3

Silicon Precursor Material

A non-limiting example of a silicon precursor material includes hexachlorodisiloxane (HCDSO). The chemical structure of HCDSO is presented below:

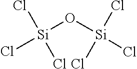

### Example 4

Silicon Precursor Material

A non-limiting example of a silicon precursor material includes hexamethyldisiloxane (HMDSO). The chemical structure of HMDSO is presented below:

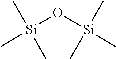

### Example 5

Deposition Rates

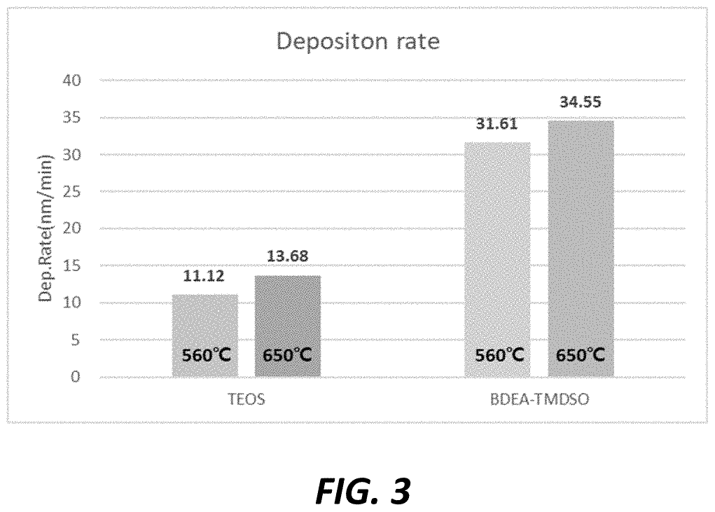

Several chemical vapor deposition processes were conducted with a control precursor material of tetraethoxysilane (TEOS) and a silicon precursor material comprising BDEA-TMDSO. The deposition rates of each of BDEA-TMDSO and TEOS at each of 560° C. and 650° C. were measured. As shown in FIG. 3, the deposition rates of BDEA-TMDSO were observed to be 2.5 times faster than the deposition rate of TEOS at 650° C. and 2.8 times faster than the deposition rate of TEOS at 560° C.

### Example 6

Silicon Precursor Material

Chemical vapor deposition processes were performed using the silicon precursor materials of Examples 1 to 4. For comparison, tetraethoxysilane (TEOS) was used as a control precursor material. All the precursor materials were deposited at a deposition temperature of 550° C. to 650° C. and a pressure of 3.5 Torr. The deposition rate and step coverage are summarized in Table 1 below.

TABLE 1
Summary of Deposition Rate and Step Coverage

| | TEOS | HCDSO | BDEA-TMDSO | HMDSO |
|---|---|---|---|---|
| Deposition Rate | 3.4 | 6.5 | 11.4 | 14.4 |
| (nm/min) | | | | |
| Step Coverage | 1x | >1.4x | >1.3x | >1.3x |

Based on the data in Table 1, the deposition rates of the silicon precursor materials was up to 4.2× greater than the deposition rate of the control precursor material. In addition, the step coverage of the silicon precursor materials was up to more than 1.4× the step coverage of the control precursor material.

What is claimed is:

1. A method for depositing a silicon precursor on a substrate, comprising:
obtaining a silicon precursor material comprising at least one siloxane linkage;
obtaining at least one co-reactant precursor material,
volatizing the silicon precursor material to obtain a silicon precursor vapor;
volatizing the at least one co-reactant precursor material to obtain at least one co-reactant precursor vapor; and
contacting the silicon precursor vapor and the at least one co-reactant precursor vapor with the substrate, under chemical vapor deposition conditions, sufficient to form a silicon-containing film on a surface of the substrate.

2. The method of claim 1, wherein the silicon precursor material comprises a compound of formula:
(A¹A²A³)Si—O—Si(B¹B²B³),
wherein each of A¹, A², A³, B¹, B², and B³ is independently a hydrogen, a halide, an alkyl, a cycloalkyl, an alkoxy, an amino, an alkylamino, an aminoalkyl, an ethynyl, an phenyl, an allyl, a vinyl, or an acetoxy.

3. The method of claim 2, wherein the halide is Cl, Br, F, or I.

4. The method of claim 2, wherein the alkyl is a linear or branched Ci to C₄ alkyl.

5. The method of claim 2, wherein the alkyl is a methyl, an ethyl, a propyl, a n-butyl, an isopropyl, a t-butyl, or a sec-butyl.

6. The method of claim 2, wherein the alkylamino has a formula:
—N(R^{a}R^{b}R^{c}),
wherein each of R^{a}, R^{b}, and R^{c} is independently a hydrogen, a methyl, an ethyl, a propyl, a n-butyl, an isopropyl, a t-butyl, or a sec-butyl.

7. The method of claim 2, wherein the alkylamino is an alkyl amine, a dialkylamine, or a trialkyl amine.

8. The method of claim 2, wherein the alkylamino is —NH(CH₂CH₃)₂ or —NHCH(CH₃)₂.

9. The method of claim 1, wherein the silicon precursor material comprises at least one of bis(diethylamino)-1,1,3,3-tetramethyldisiloxane (BDEA-TMDSO), 1,3-bis(isopropylamino)tetramethyldisiloxane (BIPA-TMDSO), or any combination thereof.

10. The method of claim 1, wherein the silicon precursor material comprises at least one of hexachlorodisiloxane, hexamethyldisiloxane, or any combination thereof.

11. The method of claim 1, wherein the silicon precursor material comprises two or more siloxane linkages.

12. The method of claim 1, wherein the chemical vapor deposition conditions comprise a deposition temperature of at least 500° C.

13. The method of claim 1, wherein the chemical vapor deposition conditions comprise a deposition temperature of at least 600° C.

14. The method of claim 1, wherein the contacting is sufficient to result in a deposition rate of 2.5 to 3 times greater than a deposition rate of a silicon precursor material control, wherein the silicon precursor material control comprises tetraethoxysilane (TEOS).

15. A precursor for chemical vapor deposition, comprising:
a silicon precursor material, wherein the silicon precursor material is a compound of formula:
(A¹A²A³)Si—O—Si(B¹B²B³),
wherein each of A¹, A², A³, B¹, B², and B³ is independently a hydrogen, a halide, an alkyl, a cycloalkyl, an alkoxy, an amino, an alkylamino, an aminoalkyl, an ethynyl, an phenyl, an allyl, a vinyl, or an acetoxy;
wherein the silicon precursor material having a reactivity with at least one co-reactant precursor material, under chemical vapor deposition conditions, sufficient to result in a silicon-containing film as a reaction product.

16. The precursor of claim 15, wherein the alkyl is a linear or branched Ci to C₄ alkyl.

17. The precursor of claim 15, wherein the alkyl is a methyl, an ethyl, a propyl, a n-butyl, an isopropyl, a t-butyl, or a sec-butyl.

18. The precursor of claim 15, wherein the alkylamino has a formula:
—N(R^{a}R^{b}R^{c}),
wherein each of R^{a}, R^{b}, and R^{c} is independently a hydrogen, a methyl, an ethyl, a propyl, a n-butyl, an isopropyl, a t-butyl, or a sec-butyl.

19. The precursor of claim 15, wherein the alkylamino is an alkyl amine, a dialkylamine, or a trialkyl amine.

20. The precursor of claim 15, wherein the alkylamino is —NH(CH₂CH₃)₂ or —NHCH(CH₃)₂.
